(19) United States
(12) Patent Application Publication
(10) Pub. No.: US 2023/0241143 A1
(43) Pub. Date: Aug. 3, 2023

(54) Method of Improving Resilience and Recovery in Healthy Mammals

(71) Applicant: Lonza Greenwood LLC, Greenwood, SC (US)

(72) Inventors: Vijaya Juturu, North Bruswick, NJ (US); Shane Durkee, Morristown, NJ (US)

(21) Appl. No.: 17/791,308

(22) PCT Filed: Jan. 27, 2021
(86) PCT No.: PCT/US2021/015170

§ 371 Date: Jul. 7, 2022

Related U.S. Application Data
(60) Provisional application No. 62/967,119, filed on Jan. 29, 2020.

(51) Int. Cl. A61K 36/05; A61P 39/06

(52) U.S. Cl. CPC A61K 36/05; A61P 39/06

(57) ABSTRACT

The present disclosure is directed to a method of improving at least one of resilience and recovery in healthy mammals undergoing intensive physical activity. The method includes administering a SOD enriched supplement to the healthy mammal during a term of intensive physical activity. The term can be long term or short term, and the mammal can be a healthy trained mammal.

### BACKGROUND

In recent years, the use of antioxidants to treat or lessen the effects of various conditions has become exceedingly popular. Particularly, antioxidants are produced by many, if not most, mammals, and help to remove reactive oxygen species from the cells of the mammal. However, the production of such antioxidants is observed to decrease as the mammal ages, often leading to an accumulation of reactive oxygen species in the body, which can cause, or worsen, many diseases associated with aging.

For instance, oxidative stress has been linked to heart disease, cancer, arthritis and other inflammatory diseases, stroke, respiratory diseases, immune deficiency, and other conditions, including Parkinson’s disease, Alzheimer’s disease, Lou Gehrig’s disease, Huntington’s disease, and Multiple Sclerosis (MS). Therefore, many supplements and nutraceuticals have been proposed that seek to supplement declining antioxidant production in an effort to mitigate or reverse the course of age related diseases.

However, while methods have been proposed for treating aging populations that suffer from diseases either due to, or worsened by, declining natural antioxidant production, a need exists for a method of treating healthy, or even trained individuals. Particularly, it has been observed that mammals undergoing time periods of intensive training, such as military personal or as observed in athletes throughout the course of the season, actually experience decrements in recovery and resilience during, and at the end of the intensive training. For instance, a study of NHL athletes was conducted, and found that the average front squat dropped from 275 pounds to 185 pounds throughout the course of the regular season. Similarly, Fry et al (1994) investigated the effects of ten-sets of one-rep-max efforts each day for two-weeks, and found that the athletes required almost eight weeks to fully recover their previous maximum strength. Furthermore, while muscle building and strengthening supplements and regimes have been proposed, no methods of supplementation have yet been provided that address these decrements observed during the course of intensified training.

Therefore, it would be a benefit to provide a method for improving muscle resilience and recovery in a mammal that is not suffering from a condition or disease. Furthermore, it would be a benefit to provide a method for improving at least one of resilience and recovery in a mammal undergoing short term intensive training or long term intensive training. Moreover, it would be a benefit to provide a method for improving at least one of term resilience and recovery in a trained mammal. Furthermore, it would be a benefit to provide a method for improving at least one of resilience and recovery in an overtrained mammal. Additionally or alternatively, it would be a benefit to provide a method for improving at least one or resilience and recovery in a mammal that has undergone or is undergoing stress induced exercise.

### SUMMARY

The present disclosure is generally directed to a method of improving at lest one of resilience and recovery in a healthy mammal. The method includes administering a SOD enriched supplement to a mammal undergoing intensive physical activity in an amount sufficient to improve at least one of resilience and recovery. In the method, the improvement in resilience and recovery is evidenced by improvement in perceived recovery, explosive strength, or time to reach peak force.

In one aspect, the SOD enriched supplement includes T. chuii. Furthermore, in an aspect, the SOD enriched supplement is a biomass of T. chuii, an extract of T. chuii, or a combination thereof.

In yet a further aspect, the heart rate of the mammal reaches about 50% to about 99% of its maximum heart rate during the intensive physical activity. Additionally or alternatively, the intensive physical activity lasts from about 2 days to about 180 days. It another aspect, the intensive physical activity lasts from about 2 days to about 14 days. Moreover, in an aspect, the intensive physical activity lasts from about 14 days to about 150 days.

Moreover, in one aspect, the SOD enriched supplement has a SOD activity of about 500 IU or greater. Furthermore, in one aspect, the oxygen radical absorbance capacity (ORAC) of the SOD enriched supplement is about 100 µmol TE/g or greater. In yet a further aspect, the oxygen radical absorbance capacity (ORAC) of the SOD enriched supplement is about 2.5 µmol TE/dose or greater.

In one aspect, the SOD enriched supplement is administered daily. Furthermore, in an aspect, the SOD enriched supplement is administered once per day. Additionally or alternatively, the SOD enriched supplement is administered from 2 to 5 times per day, where the total ORAC of the SOD enriched supplement is about 2.5 µmol TE or greater per day. Furthermore, in one aspect, the SOD enriched supplement is administered in an amount of about 1 mg to about 100 mg per administration.

In a further aspect, the SOD enriched supplement further includes at least one of a vitamin, a mineral, an essential fatty acid, or an amino acid. Moreover, in an aspect, the SOD enriched supplement includes vitamin E, vitamin C, or a combination thereof. Additionally or alternatively, in an aspect, the SOD enriched supplement includes potassium, magnesium, calcium, zinc, or combinations thereof.

In one aspect, an improvement in at least one of resilience and recovery is evidenced by recovery after intensive physical activity to a non-significant difference from the mammals pre-intensive physical activity explosive strength (measured as its mean rate of force development) in about 96 hours or less. Furthermore, in an aspect, an improvement in at least one of resilience and recovery is evidenced by no significant decrease in time to peak force within about 24 hours to about 96 hours after intensive physical activity. Additionally or alternatively, in one aspect, an improvement in at least one or resilience and recovery is evidenced by an improvement in perceived recovery as compared to a mammal who has not undergone SOD enriched supplement administration.

Moreover, in yet a further aspect, the mammal has an age of about 55% or less of its expected life span. In another aspect, the mammal has an age of about 60% or more of its expected life span.

Furthermore, in one aspect, the SOD enriched supplement includes a blend or microalgae. In one aspect, the blend of microalgae includes T. chuii.

### BRIEF DESCRIPTION OF THE DRAWINGS

A full and enabling disclosure of the present disclosure is set forth more particularly in the remainder of the specification, including reference to the accompanying figures, in which:

FIG. 1 shows a graph of mean explosive strength according to an aspect of the present disclosure;

FIG. 2 shows a graph of change in mean explosive strength according to an aspect of the present disclosure’

FIG. 3 shows a graph of salivary immunoglobin A levels according to an aspect of the present disclosure;

FIG. 4 shows a graph of time to peak force according to an aspect of the present disclosure;

FIG. 5 shows a graph of mean explosive strength according to an aspect of the present disclosure; and

FIG. 6 shows a graph of change in perceived recovery according to an aspect of the present disclosure.

### DEFINITIONS

As used herein, the terms “about,” “approximately,” or “generally,” when used to modify a value, indicates that the value can be raised or lowered by 10% and remain within the disclosed aspect.

The term “therapeutically effective amount as used herein, with, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “supplement” means a product in addition to the normal diet of the mammal but may be combined with a mammal’s normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “nutraceutical” and “ingredient” refers to any compound added to a dietary source (e.g., a fortified food or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved resilience and recovery and can include without limitation canine, equine, feline, bovine, ovine, human, or porcine mammals.

As used herein, “healthy” refers to the absence of illness or injury.

The term “intensive physical activity” means activity that lasts about 20 minutes or more, such as about 25 minutes or more, such as about 30 minutes or more, such as at least about 45 minutes or more, and where the heart rate of the mammal reaches about 50% to about 99% of its maximum heart rate, such as about 55% to about 95%, such as about 60% to about 90%, such as about 70% to about 85% of the maximum heart rate of the mammal.

As used herein, “trained” refers to a mammal that undergoes physical activity that lasts about 30 minutes where the heart rate is greater than 75% of the mammals HR max, three days per week, and that has at least one year of resistance training experience.

As used herein, “overtrained” or “overtraining”, refers to a mammal that has reached an imbalance of training or stress and recovery that result in a decrement in fitness level, and may also be referred to as overtraining syndrome, but that does not rise to the level of injury (which may also be referred to as acute overtraining).

As used herein, “Delivery” refers to approaches, formulations, technologies, and systems for transporting a compound in the body and/or into the body, including the use of nanoparticles, as needed to achieve the compounds desired therapeutic effect and/or nutraceutical effect, as a standalone compound or in combination. The delivery may be controlled release, short acting, and/or long lasting release, not limited to the following:
Immediate release: Coni-Snap® Gelatin, Vcaps® Plus, Plantcaps®, Vcaps® Gen C;
Modified release: Vcaps® Enteric, DRcaps™, Coni-Snap® Gelatin and Coni-Snap® Gelatin-PEG, Vcaps® and Vcaps® Plus;
PCcaps®, DBcaps®, Colorista®, End to End: Coni-Snap® Sprinkle, Press-Fit®, Xpress-Fit®, Licaps®;
Furthermore, in one or more aspects, delivery may also include bioavailability enhancing technologies not limited to:
Liquid filled hard capsules: CFS1200™, CFS1500™, LEMS®70
Micronization and containment equipment; and Technologies for clinical development and small batches, not limited to:
   Powder micro-dosing systems: Xcelolab®, Xcelodose® 120S, Scelodose® 600S;
   Hard capsule filing machines: Ultra III™.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a method of improving resilience and recovery in healthy mammals undergoing intensive physical activity or overtraining . For instance, the present disclosure has found that by administering a superoxide dismutase enriched supplement to a mammal undergoing intensive physical activity or overtraining for a period of time, decreases in recovery and resiliency, such as immune function, explosive strength, and perceived recovery were improved, or eliminated. Furthermore, the present disclosure has found that administration of a superoxide dismutase enriched supplement containing a high superoxide dismutase activity, during short or long term periods of intensive physical activity or overtraining, may further improve resilience and recovery, and can further promote breakdown of radical oxygen species produced during intensive exercise, as well as improve mitochondrial biogenesis and increase glucose uptake by muscles.

Furthermore, the present disclosure has found that the SOD enriched supplement may also improve decrements observed during exercise induced stress or overtraining. Particularly, the SOD enriched supplement of the present disclosure may help to limit decreases in immune function observed during periods of intensive physical activity that may rise to the level of stress induced exercise or overtraining. For instance, overtraining and exercise induced stress may mobilize natural killer cells, lymphocytes, proinflammatory cytokines, and stress hormones, which can suppress immune cell function. Surprisingly, the present disclosure has found that the SOD enriched supplement may lessen decreases in salivary immunoglobin A observed in subjects that have undergone intensive physical activity and/or overreaching, showing improvement in decrements associated with overtraining and exercise induced stress.

Thus, in one aspect, the present disclosure has found that a superoxide dismutase (SOD) enriched supplement containing a SOD activity of about 500 IU per administration or more, such as about 525 IU or more, such as about 550 IU or more, such as about 575 IU or more, such as about 600 IU or more, such as about 625 IU or more, such as about 650 IU or more, such as about 675 IU or more, such as about 700 IU or more, such as about 725 IU or more, such as about 750 IU or more, such as about 775 IU or more, such as about 800 IU or more, such as about 825 IU or more, such as about 850 IU or more, such as about 875 IU or more, such as about 900 IU or more, such as about 925 IU or more, such as about 950 IU or more, such as about 975 IU or more, such as about 1000 IU or more, such as about 1025 IU or more, such as about 1050 IU or more per administration, such as up to about 2000 IU or less per administration, can improve resilience and recovery after long term and/or short term intensive physical activity in healthy mammals. Thus, in one aspect, the SOD enriched supplement is administered to the mammal in an amount sufficient to improve at least one of resiliency and recovery after intensive physical activity.

Furthermore, in one aspect, the SOD enriched supplement may have an SOD activity of about 15,000 IU/g or greater, such as about 20,000 IU/g or greater, such as about 25,000 IU/g or greater, such as about 30,000 IU/g or greater, such as about 35,000 IU/g or greater, such as about 40,000 IU/g or greater, such as about 42,500 IU/g or greater, up to a SOD activity of about 55,000 IU/g or less.

In one aspect, the SOD enriched supplement may be a SOD enriched marine phytoplankton, and may therefore be natural, non-GMO, and vegan. In one such aspect, the marine phytoplankton may be enriched with SOD by culturing under abiotic stress, or any other method known in the art sufficient to yield a SOD activity as described above. In a further aspect, the SOD enriched phytoplankton may be of the species Tetraselmis chuii, which has been found to be susceptible to high levels of SOD enrichment. Thus, in one aspect, the SOD enriched supplement is a biomass of T. chuii enriched with SOD, or an extract of the SOD enriched T. chuii, such as a protein extract, or a combination thereof. For instance, in one such aspect, the SOD enriched T. chuii may be available under the trade name Oceanix™. However, in a further aspect, the SOD enriched supplement may be a blend of marine phytoplankton, where at least a portion of the blend are T. chuii.

Notwithstanding the SOD enriched supplement selected, the SOD enriched supplement may also have a large oxygen radical absorbance capacity (ORAC), as measured according to ORAC 6.0. Particularly, ORAC tests measure antioxidant scavenging activity against oxygen radicals that are known to be involved in the pathogenesis of aging and common disease, and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as µmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2.2’ azobis(2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosyndnonimine hydrochloride. Finally, the ORAC assay against superoxide measures the antioxidant capacity of the sample to protect hydroethidine from damage by a superoxide which is generated from xanthine oxidase.

Thus, in one aspect, the SOD enriched supplement according to the present disclosure may have a total ORAC of about 100 µmol TE/g or greater, such as about 150 µmol TE/g or greater, such as about 200 µmol TE/g or greater, such as about 250 µmol TE/g or greater, such as about 300 µmol TE/g or greater, such as about 350 µmol TE/g or greater, such as about 400 µmol TE/g or greater, such as about 450 µmol TE/g or greater, such as about 500 µmol TE/g or greater, such as about 550 µmol TE/g or greater, such as about 565 µmol TE/g or greater, up to about 750 µmol TE/g, or any ranges or values therebetween.

Furthermore, in one aspect, the SOD enriched supplement according to the present disclosure may have a ORAC against peroxyl radicals of about 10 µmol TE/g or greater, such as about 15 µmol TE/g or greater, such as about 20 µmol TE/g or greater, such as about 25 µmol TE/g or greater, such as about 30 µmol TE/g or greater, up to about 50 µmol TE/g, or any ranges or values therebetween.

Similarly, in one aspect, the SOD enriched supplement according to the present disclosure may have a ORAC against hydroxyl radicals of about 50 µmol TE/g or greater, such as about 75 µmol TE/g or greater, such as about 100 µmol TE/g or greater, such as about 125 µmol TE/g or greater, such as about 150 µmol TE/g or greater, such as about 175 µmol TE/g or greater, up to about 250 µmol TE/g, or any ranges or values therebetween.

Additionally or alternatively, in one aspect, the SOD enriched supplement according to the present disclosure may have a ORAC against peroxynitrite of about 1 µmol TE/g or greater, such as about 2.5 µmol TE/g or greater, such as about 5 µmol TE/g or greater, such as about 7.5 µmol TE/g or greater, such as about 8 µmol TE/g or greater, up to about 15 µmol TE/g, or any ranges or values therebetween.

In one aspect, the SOD enriched supplement according to the present disclosure may have a ORAC against superoxide anion of about 150 µmol TE/g or greater, such as about 200 µmol TE/g or greater, such as about 250 µmol TE/g or greater, such as about 300 µmol TE/g or greater, such as about 325 µmol TE/g or greater, such as about 350 µmol TE/g or greater, up to about 450 µmol TE/g, or any ranges or values therebetween.

Thus, in one aspect, the SOD enriched supplement may be administered so as to have a total ORAC capacity per administration of about 2.5 µmol TE/ administration or greater, such as about 3 µmol TE/administration or greater, such as about 3.5 µmol TE/administration or greater, such as about 4 µmol TE/administration or greater, such as about 4.5 µmol TE/administration or greater such as about 5 µmol TE/administration or greater, such as about 5.5 µmol TE/ administration or greater, such as about 6 µmol TE/administration or greater, such as about 6.5 µmol TE/administration or greater, such as about 7 µmol TE/administration or greater, such as about 7.5 µmol TE/administration or greater, such as about 8 µmol TE/administration or greater, such as about 8.5 µmol TE/administration or greater, such as about 9 µmol TE/administration or greater, such as about 9.5 µmol TE/administration or greater, such as about 10 µmol TE/administration or greater, such as about 10.5 µmol TE/administration or greater, such as about 11 µmol TE/administration or greater, such as about 11.5 µmol TE/administration or greater, such as about 12 µmol TE/administration or greater, such as about 12.5 µmol TE/administration or greater, such as about 13 µmol TE/administration or greater, such as about 13.5 µmol TE/administration or greater, such as about 14 µmol TE/administration or greater, such as about 20 µmol TE/administration or less.

For instance, in one aspect, the SOD enriched supplement may be administered to the mammal in an amount of about 1 mg to about 100 mg per administration, such as about 5 mg to about 75 mg per administration, such as about 10 mg to about 50 mg per administration, such as about 15 mg to about 40 mg per administration, such as about 20 mg to about 30 mg per administration, or any ranges or values therebetween.

In one aspect, the SOD enriched supplement is administered once per day, twice per day, three times per day, up to about five times per day. However, in one aspect, the SOD enriched supplement is administered once per day.

Additionally or alternatively, the SOD enriched supplement may be administered more than once per day such that the total number of administrations delivers about 2.5 µmol TE or greater, such as about 3 µmol TE or greater, such as about 3.5 µmol TE or greater, such as about 4 µmol TE or greater, such as about 4.5 µmol TE or greater such as about 5 µmol TE or greater, such as about 5.5 µmol TE or greater, such as about 6 µmol TE or greater, such as about 6.5 µmol TE or greater, such as about 7 µmol TE or greater, such as about 7.5 µmol TE or greater, such as about 8 µmol TE or greater, such as about 8.5 µmol TE or greater, such as about 9 µmol TE or greater, such as about 9.5 µmol TE or greater, such as about 10 µmol TE or greater, such as about 10.5 µmol TE or greater, such as about 11 µmol TE or greater, such as about 11.5 µmol TE or greater, such as about 12 µmol TE or greater, such as about 12.5 µmol TE or greater, such as about 13 µmol TE or greater, such as about 13.5 µmol TE or greater, such as about 14 µmol TE or greater, such as about 20 µmol TE or less.

Furthermore, SOD enriched supplement may also include various other components that provide a further benefit to the healthy mammal.

For instance, the SOD enriched supplement and/or the composition may contain one or more vitamins, minerals, essential fatty acids, amino acids, polyphenols stilbenoids, curcumininoids, tannins, flavones, flavonols, flavan-3-ols, flavanones, anthocyanidins, anthocyanins, isoflavones, flavanonols, proanthocyanidins, dihydroxybenzoic acids, carotenoids, and pyridine alkaloids. In one aspect, the composition may contain an effective amount of an antioxidant, an amino acid, an essential fatty acid, a polyphenol, or combinations thereof.

For example, in one aspect the SOD enriched supplement contains at least one vitamin, such as at least one of vitamin B, vitamin C, and vitamin E. Vitamins may be contained in the SOD enriched supplement in an amount of from about 50 ug/g of supplement to about 5000 ug/g, such as about 100 ug/g to about 4500, such as about 250 ug/g to about 4000 ug/g, such as about 400 ug/g to about 3500 ug/g, or any ranges or values therebetween. The above ranges may be for any one vitamin alone or a total amount of all vitamins. In one aspect, vitamin E is present in the SOD enriched supplement in an amount of about 100 ug/g to about 1000 ug/g, such as about 250 ug/g to about 750 ug/g, such as about 400 ug/g to about 600 ug/g, or any ranges or values therebetween. In another aspect, vitamin C is present in the SOD enriched supplement in an amount of about 1000 ug/g to about 5000 ug/g, such as about 2000 ug/g to about 4000 ug/g, such as about 3000 ug/g to about 3750 ug/g, or any ranges or values therebetween.

Furthermore, in an aspect, the SOD enriched supplement contains at least one mineral, such as at least one of potassium magnesium, zinc or calcium. Minerals may be contained in the SOD enriched supplement in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the SOD enriched supplement contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the SOD enriched supplement contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the SOD enriched supplement contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

Additionally, the SOD enriched supplement may further include at least one additive that enhances sports performance or that further contributes to reducing oxidative stress. For instance, in one aspect, an additive may be one or more of curcumin, spirulina, astaxanthin, or other carotenoids. Furthermore, in one aspect, the present disclosure may include one or more microalgae with high SOD and ORAC levels, where the one or more microalgae are different than a microalgae selected as the SOD enriched supplement. Particularly, such microalgae may further help to reduce oxidative stress, and may contribute further anti-inflammatory properties and protection against infections, including improvement in immune health. Moreover, in one aspect, an additive may include one or more probiotics.

It should be understood that any further components may be additives to the supplement, or may be contained in the SOD activity providing component. For instance, in one aspect, a phytoplankton is selected and/or enriched to provide one or more vitamins and/or minerals in addition to the SOD activity discussed herein.

Furthermore, the present disclosure has found that it may also be beneficial if the SOD enriched supplement has a structure containing a high percentage of protein. Thus, in one aspect, the SOD enriched supplement is about 5% to about 75% protein by weight of the supplement, such as about 10% to about 70%, such as about 15% to about 65%, such as about 20% to about 60%, such as about 25% to about 55%, such as about 30% to about 50% protein by weight, or any ranges or values therebetween.

Notwithstanding the final composition of the SOD enriched supplement selected, the present disclosure has found that the SOD enriched supplement improves resilience and recovery during and/or after both short term and long term intensive physical activity . Thus, in one aspect, the intensive physical activity may last about 2 days or more, such as about 5 days or more, such as about 7 days or more, such as about 10 days or more, such as about 14 days or more, such as about 21 days or more, such as about 28 days or more, such as about 35 days or more, such as about 42 days or more, such as about 49 days or more, such as about 56 days or more, such as about 63 days or more, such as about 70 days or more, up to about 180 days, and the SOD enriched supplement may be administered at least once on each day of the term. Of course, it should be understood that long term may be greater than 180 days based upon some sports and training regimes.

Regardless, in one aspect, active training rising to the level of intensive physical activity may take place on about 50% or more of the respective days, such as about 60% or more, such as about 70% or more of the respective days of the term (e.g. during a time period of 7 days, training may take place on 5 days), and still be considered to rise to the level of intensive physical activity for the entirety of the term, or, in one aspect, intensive physical activity may take place on all days of the term.

Nonetheless, in one aspect, the term may be considered to be short term intensive physical activity, and lasts from about 2 days to about 21 days, such as about 5 days to about 14 days, or any ranges or valued therebetween. Alternatively, the term may be considered to be long term intensive physical activity, and lasts from about 14 days to about 150 days, such as about 21 days to about 100 days, such as about 28 days to about 48 days, or any ranges or values therebetween.

Notwithstanding the final composition of the SOD enriched supplement, in one aspect, a mammal being administered the SOD enriched supplement while undergoing intensive physical activity according to the present disclosure can exhibit recovery of the mammals explosive strength (measured as its mean rate of force development) after intensive physical activity in about 96 hours or less, such as about 72 hours or less, such as about 60 hours or less, such as about 48 hour or less, where recovery is measured as returning to a value within about 10% of the original (pre-intensive physical activity) value, such as about 7.5 %, such as about 5%, such as about 2.5%, such as about 1%, and, in one instance, may return to a value where statistically p<0.05. Where intensive physical activity may be confirmed to have occurred by significant decreases in explosive strength during intensive physical activity, which will be discussed in greater detail in the examples below.

Additionally or alternatively, the mammal may exhibit recovery as defined above within about 168 hours or less, such as about 144 hours or less, such as about 120 hours or less, even when significant changes in explosive strength are observed.

Furthermore, a mammal being administered the SOD enriched supplement while undergoing intensive physical activity according to the present disclosure can exhibit or experience a perceived recovery of about 10% or greater than pre-administration perceived recovery, such as about 12.5% or greater, such as about 15% or greater than pre-administration perceived recovery.

In one aspect, a mammal being administered the SOD enriched supplement while undergoing intensive physical activity according to the present disclosure may not experience or exhibit a significant decrease in immune function, measured as a decrease in salivary immunoglobin A(IgA). For instance, in one aspect, the mammal may have a salivary IgA within about 24 hours to about 96 hours after intensive physical activity, such as about 36 hours to about 72 hours, such as about 48 hours after intensive physical activity within about 10% of the original (pre intensive physical activity) value, such as about 7.5 %, such as about 5%, such as about 2.5%, such as about 1%, and, in one instance, may return to a value where statistically p<0.05.

Moreover, in an aspect, a mammal being administered the SOD enriched supplement while undergoing intensive physical activity according to the present disclosure may not experience or exhibit a significant decrease in time to peak force after the intensive physic activity, or such as within about 24 hours to about 96 hours after intensive physical activity, such as about 36 hours to about 72 hours, such as about 48 hours after intensive physical activity such that the time to peak force may be within about 10% of the original (pre intensive physical activity) value, such as about 7.5 %, such as about 5%, such as about 2.5%, such as about 1%, and, in one instance, may return to a value where statistically p<0.05.

Furthermore, in one aspect, the SOD enriched supplement can be administered orally as a solid, liquid, suspension, or gas. The composition may be administered via buccal or sublingual administration. In one embodiment, the composition may be administered as a capsule, tablet, caplet, pill, troche, drop, lozenge, powder, granule, syrup, tea, drink, thin film, seed, paste, herb, botanical, and the like.

In addition to being administered orally, the the SOD enriched supplement can also be administered using other routes including intranasal, intravenous, intramuscular, intragastric, and the like.

When the SOD enriched supplement is combined with a food or beverage composition, the food or beverage composition may comprise any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, or the like.

The SOD enriched supplement of the present disclosure may further comprise one or more excipients as further additives in the composition. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, and waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one embodiment, the SOD enriched supplement of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the SOD enriched supplement in an amount sufficient to reduce the hydroscopic properties of the SOD enriched supplement and/or prevent the SOD enriched supplement from absorbing moisture. A stabilizer package may also be combined with the composition in order to improve the handling properties of the SOD enriched supplement. For instance, the stabilizer package may allow the SOD enriched supplement to have better flow properties, especially when in granular form.

In one embodiment, the SOD enriched supplement may be combined with a polymer binder in conjunction with a stabilizer package. In addition, a coating material may also be applied to the SOD enriched supplement after the SOD enriched supplement has been combined with the polymer binder and the stabilizer package. The coating material, for instance, may contain at least one fat. In accordance with the present disclosure, the above components can be added to any suitable pharmaceutical composition in addition to the SOD enriched supplement of the present disclosure. For instance, the above components may be added to any pharmaceutical composition containing a carnitine or an amino acid.

The polymer binder and the stabilizer package may be combined with the SOD enriched supplement in a manner that homogeneously incorporates the stabilizer package into the product. In one embodiment, for instance, the SOD enriched supplement of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Ltd.

Once the polymer binder is combined with the SOD enriched supplement such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the SOD enriched supplement. For instance, the stabilizer package has been found to stabilize the SOD enriched supplement and make the SOD enriched supplement less hydroscopic. The composition is also easier to handle and, when in granular form, produces a free-flowing product.

The oxide particles that may be added to the SOD enriched supplement may comprise silica. For instance, the oxide particles may comprise precipitated silica particles. The silica particles may have a particle size (d50, laser diffraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the SOD enriched supplement in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the SOD enriched supplement in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the SOD enriched supplement may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the SOD enriched supplement in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Once the above components are combined together to form the SOD enriched supplement, the SOD enriched supplement can optionally be combined with a coating material. In one embodiment, for instance, the SOD enriched supplement may comprise a granular composition to which a coating material is applied that contains a fat. The coating material, for instance, may comprise a hydrogenated oil, such as hydrogenated palm oil. In one particular embodiment, the coating material may comprise hydrogenated palm oil combined with palm stearine. In one embodiment, the hydrogenated oil may be present in the pharmaceutical composition in an amount from about 5% to about 35% by weight. The palm stearine, on the other hand, may be present in the pharmaceutical composition in an amount from about 2% to about 10% by weight. When present together, the weight ratio between the hydrogenated palm oil and the palm stearine may be from about 10:1 to about 1:1, such as from about 6:1 to about 2:1. In one embodiment, the hydrogenated palm oil and the palm stearine are present at a weight ratio of about 4:1.

Furthermore, the mammal undergoing administration in accordance with the present disclosure can comprise any suitable mammal. For instance, the mammal may be human or canine. The composition can be fed to a mammal of any age such as from parturition through the adult life in the mammal. In various embodiments the mammal may be a human, dog, a cat, a horse, a pig, a sheep, or a cow. In many embodiments, the mammal can be in early to late adulthood. For instance, the active mammal may have an age that is at least 10%, such as least 15%, such as least 20%, such as least 25%, such as least 30%, such as least 35%, such as least 40%, such as least 45%, such as least 50%, such as least 55%, such as least 60%, such as least 65%, such as least 70%, such as least 75%, such as least 85%, such as least 90%, such as least 95% of its expected life span. The mammal may have an age such that it is less than about 95%, such as less than about 90%, such as less than about 85%, such as less than about 80%, such as less than about 75%, such as less than about 70%, such as less than about 65%, such as less than about 60%, such as less than about 55%, such as less than about 50%, such as less than about 45%, such as less than about 40%, such as less than about 35%, such as less than about 30%, such as less than about 25%, such as less than about 20%, such as less than about 15%, such as less than about 10% of its expected life span. A determination of life span may be based on actuarial tables, calculations, or the like.

Nonetheless, certain embodiments of the present disclosure may be better understood according to the following examples, which are intended to be non-limiting and exemplary in nature.

### Example 1

Twenty trained male and female subjects were randomly divided into a microcrystalline cellulose based placebo (PLA,) or SOD enriched supplement (Oceanix™ OCX, 25 mg per day) condition for 14 days. Subjects were all tested at baseline and then placed on an intensified training protocol for 5 days and retested 24 and 48 hours following. Time to reach Peak Force and Explosive Strength, measured as rate of force development during an isometric mid-thigh pull were assessed. Immune function was determined using Salivary IgA. Repeated measures ANOVA and Tukey post hoc analysis were used to assess differences.

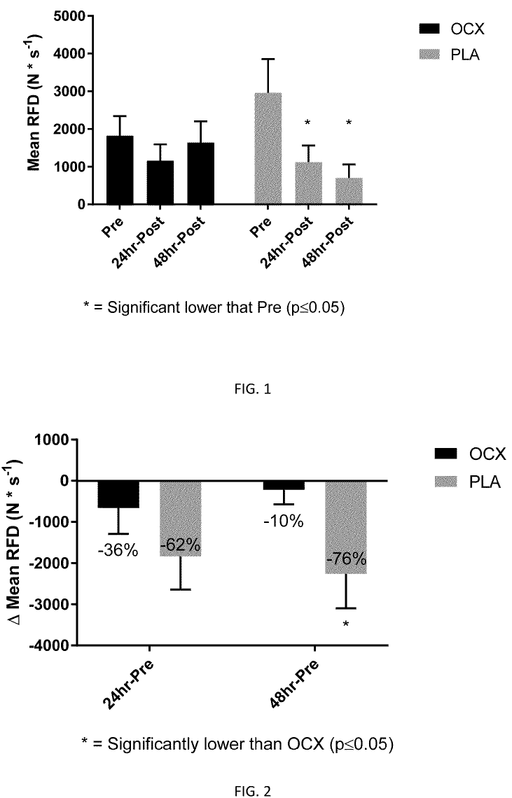

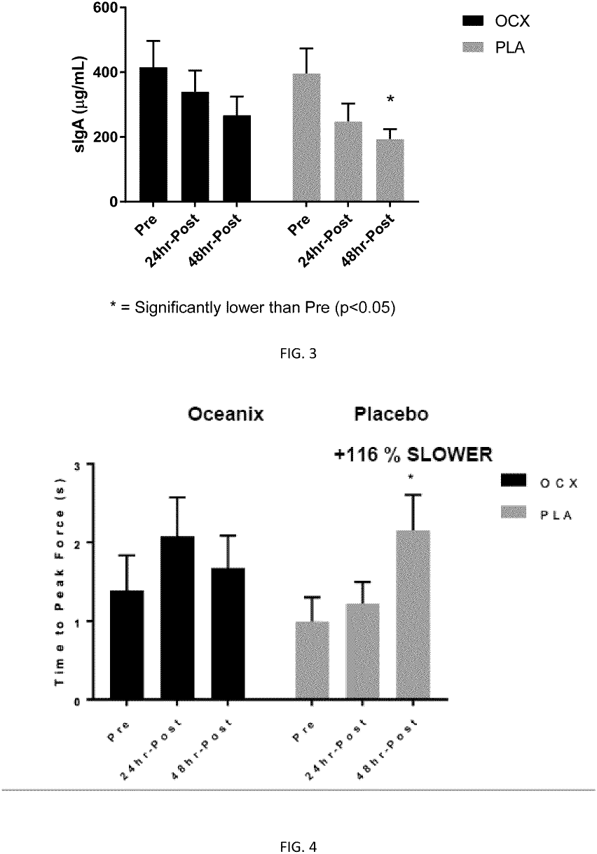

Referring to FIGS. 1-4, no statistical differences for baseline values were found between groups on any variable. Explosive Strength was significantly lower at 24 (-62 %) and 48 (-78 %) hours in the PLA but not OCX, compared to pre levels (where significance is based upon p<0.05). Additionally, the absolute mean difference in Explosive Strength from Pre to 48 hr-Post was significantly lower in PLA (p<0.05, meandiff: -2041, 95%CI: -3899 to -183 N * s-1, FIG. 1) compared to OCX, indicating greater recovery in OCX. Furthermore, post IgA levels decreased compared to Pre (-178%) in the PLA, but not in the OCX group compared to baseline at 48 hours. Finally, time to peak force values were slower at 48 hr-Post in the PLA, but not the OCX group as compared to the baseline at 4 hours.

As shown, fourteen days of administration of a SOD enriched supplement (Oceanix™ ) during short periods of intensified training shows increased resilience and better recovery from vigorous exercise as compared to a mammal who has not been administered a SOD enriched supplement.

### Example 2

Twenty trained male and female subjects were randomly divided into placebo (PLA) or SOD enriched supplement (Oceanix™ OCX, 25 mg per day) conditions. The subjects were all placed on a 6-week resistance training program interspersed with purposeful overreaching on weeks 2 and 5, and low volume recovery training on week 6. Explosive Strength, measured as mean rate of force development (Mean RFD) during the take-off phase of a countermovement jump, was assessed at pre-test, and at weeks 2, 5 and 6. Perceived recovery scale (PRS) changes (0-10) relative to initial maintenance training levels were taken on each successive week from baseline.

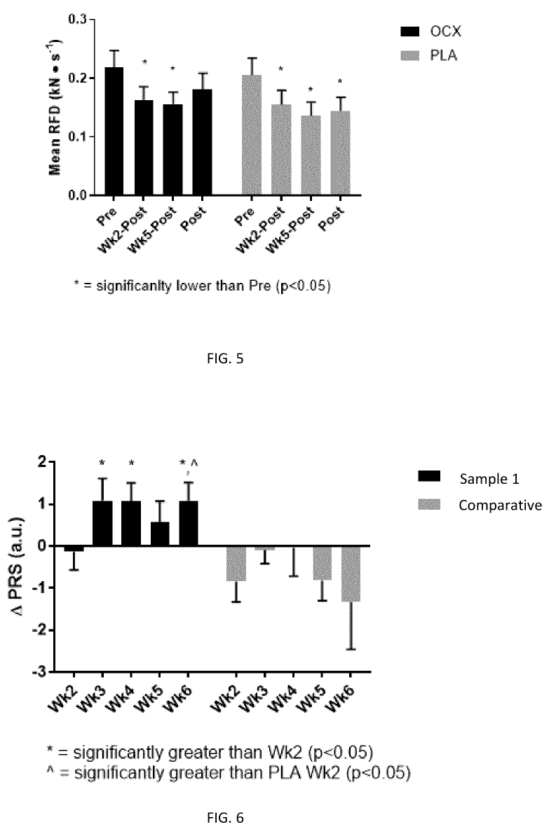

Referring to FIGS. 5 and 6, significant within-group differences for explosive strength during the takeoff phase of the countermovement jump were detected. Specifically, mean explosive strength was significantly lower at Wk2-Post (OCX: p<0.05, mean_{diff} = -0.056 kN • s⁻¹; PLA: p<0.05, mean_{diff} = -0.051 kN • s⁻¹) and Wk5-Post (OCX: p<0.05, mean_{diff} = -0.063 kN • s⁻¹; PLA: p<0.05, mean_{diff} = -0.069 kN • s⁻¹) compared to Pre for both groups indicating overreaching had occurred. While Wk6 levels compared to Pre were still depressed in PLA (p<0.05, mean_{diff} = -0.061 kN • s⁻¹), OCX had recovered (i.e. not substantially/statistically different from Pre-activity levels).

Analysis of relative perceived recovery (PRS) scale change indicated significant within-group differences for OCX whereby PRS levels at Wk3 (p<0.05, Δ +1.1, +16%), Wk4 (p<0.05, Δ +1.1, +16%), and Wk6 (p<0.05, Δ +1.1, +16%) were greater than Wk2 levels (p<0.05). Additionally, relative change in PRS at Wk6 was significantly greater for OCX at Wk6 compared to PLA (p<0.05; OCX: 1.1 ± 0.4 a.u., +16%; PLA: -0.5 ± 0.9 a.u, -6%).

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method of improving at least one of resilience and recovery in a healthy mammal, comprising
administering a SOD enriched supplement to a mammal undergoing intensive physical activity in an amount sufficient to improve the at least one of resilience and recovery,
wherein the improvement in resilience and recovery is evidenced by improvement in perceived recovery, explosive strength, or time to reach peak force.

2. The method of claim 1, wherein the SOD enriched supplement comprises T. chuii.

3. The method of claim 2, wherein the SOD enriched supplement is a biomass of T. chuii, an extract of T. chuii, or a combination thereof.

4. The method of claim 1, wherein the heart rate of the mammal reaches about 50% to about 99% of its maximum heart rate during the intensive physical activity.

5. The method of claim 1, wherein the intensive physical activity lasts from about 2 days to about 180 days.

6. The method of claim 5, wherein the intensive physical activity lasts from about 2 days to about 14 days.

7. The method of claim 5, wherein the intensive physical activity lasts from about 14 days to about 150 days.

8. The method of claim 1, wherein the SOD enriched supplement has a SOD activity of about 500 IU or greater.

9. The method of claim 1, wherein an oxygen radical absorbance capacity (ORAC) of the SOD enriched supplement is about 100 µmol TE/g or greater.

10. The method of claim 1, wherein an oxygen radical absorbance capacity (ORAC) of the SOD enriched supplement is about 2.5 µmol TE/dose or greater.

11. The method of claim 1, wherein the SOD enriched supplement is administered daily.

12. The method of claim 11, wherein the SOD enriched supplement is administered once per day.

13. The method of claim 11, wherein the SOD enriched supplement is administered from 2 to 5 times per day, wherein the total ORAC of the SOD enriched supplement is about 2.5 µmol TE or greater per day.

14. The method of claim 1, wherein the SOD enriched supplement is administered in an amount of about 1 mg to about 100 mg per administration.

15. The method of claim 1, wherein the SOD enriched supplement further includes at least one of a vitamin, a mineral, an essential fatty acid, or an amino acid.

16. The method of claim 15, wherein the SOD enriched supplement includes vitamin E, vitamin C, or a combination thereof.

17. The method of claim 15, wherein the SOD enriched supplement includes potassium, magnesium, calcium, zinc, or combinations thereof.

18. The method of claim 1, wherein an improvement in at least one of resilience and recovery is evidenced by recovery after intensive physical activity to a non-significant difference from the mammals pre-intensive physical activity explosive strength (measured as its mean rate of force development) in about 96 hours or less.

19. The method of claim 1, wherein an improvement in at least one of resilience and recovery is evidenced by no significant decrease in time to peak force within about 24 hours to about 96 hours after intensive physical activity.

20. The method of claim 1, wherein an improvement in at least one or resilience and recovery is evidenced by an improvement in perceived recovery as compared to a mammal who has not undergone SOD enriched supplement administration.

21. The method of claim 1, wherein the mammal has an age of about 55% or less of its expected life span.

22. The method of claim 1, wherein the mammal has an age of about 60% or more of its expected life span.

24. The method of claim 1, wherein the SOD enriched supplement includes a blend of microalgae.

25. The method of claim 24, wherein the blend of microalgae includes T. chuii.
